(19) United States
(12) Patent Application Publication
(10) Pub. No.: US 2021/0299147 A1
(43) Pub. Date: Sep. 30, 2021

(54) SLEEP AID AND RELATED METHODS

(71) Applicant: John Frogge, Lakewood, CO (US)

(72) Inventor: John Frogge, Lakewood, CO (US)

(21) Appl. No.: 17/197,395

(22) Filed: Mar. 10, 2021

Related U.S. Application Data
(60) Provisional application No. 62/994,918, filed on Mar. 26, 2020.

(51) Int. Cl. A61K 31/593; A61K 31/4045; A61K 31/355; A61K 31/122; A61K 31/197; A61K 33/06; A61K 31/405; A23L 33/00; A23L 29/00

(52) U.S. Cl. CPC A61K 31/593; A61K 31/4045; A61K 31/355; A61K 31/122; A61K 31/197; A23L 29/015; A61K 31/405; A23L 33/30; A23L 29/035; A23L 29/045; A61K 33/06

(57) ABSTRACT

A stable composition suitable for use as a sleep aid is described. The composition has, in combination: cannabinol, melatonin, magnesium glycinate, menaquinone, cholecalciferol, calcium, Vitamin E, L-Tryptophan, and gamma-aminobutyric acid.

### CROSS-REFERENCE TO RELATED APPLICATIONS

This application claims priority to U.S. Provisional Application No. 62/994,918, filed Mar. 26, 2020 and entitled “SLEEP AID AND RELATED METHODS,” the entire disclosure of which is hereby incorporated by reference for all proper purposes.

### FIELD

This invention is related to products for human consumption. Specifically, but not intended to limit the invention, embodiments of the invention are related to sleep aids.

### BACKGROUND

It is estimated that approximately 60 million people in the United States and many more worldwide currently suffer from insomnia.

Over the years, countless sleep aids have been developed, from prescription-strength formulas manufactured by pharmaceutical companies to home-grown recipes developed by laypersons and sufferers alike.

More recently, formulas containing CBD, or cannabidiol, which is one of the primary active compounds, or cannabinoids, found in cannabis plants, have been made available.

There remains a need, however, to provide an effective sleep aid and other new and useful products and methods.

### SUMMARY

An exemplary stable composition suitable for use as a sleep aid has, in combination: cannabinol, melatonin, magnesium glycinate, menaquinone, cholecalciferol, calcium, Vitamin E, L-Tryptophan, and gamma-aminobutyric acid.

### BRIEF DESCRIPTION ON THE DRAWINGS

FIG. 1 is a listing of ingredients in an exemplary composition.

### DETAILED DESCRIPTION

Before describing embodiments herein in detail, it is expedient to further describe some of the problems facing sufferers of insomnia and those attempting to provide solutions. One concern is that of addiction. Specifically, it is a challenge to provide an effective sleep aid to enable healthy sleep, while also being non habit-forming and non-addictive. To address this problem, the Applicant has developed a composition that calms the mind and helps induce sleep in a manner that is not suggested by known products. Critically, it is noted herein that the combination of chemicals or ingredients in the claimed composition provide a synergistic effect and unexpected result, because none of the chemicals alone provide the benefits seen by the claimed composition. The claimed composition as a whole provides effects that are greater than the sum of the effects of the parts.

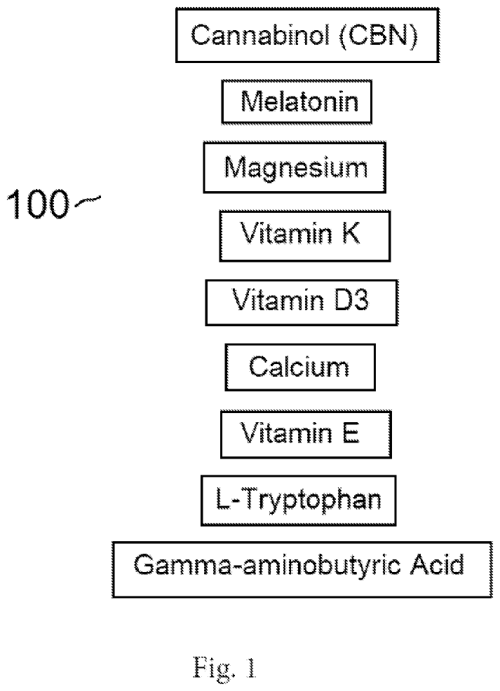

With reference now to FIG. 1, a stable composition 100 suitable for use as a sleep aid is described herein. The composition 100 includes, in combination, cannabinol, melatonin, magnesium or magnesium glycinate, menaquinone or long-chain menaquinone or Vitamin K or Vitamin K7, cholecalciferol or Vitamin D3, calcium, Vitamin E or alpha-tocopherol, L-tryptophan, and gamma-aminobutyric acid. The composition 100 may include a stable carrier or suspension.

The composition 100 may have a multiple of the following formula:

Cannabinol: between 10 micrograms and 150 milligrams;

Melatonin: between 0.1 milligrams and 9 milligrams;

Magnesium Glycinate: between 1 milligram and 100 milligrams;

Menaquinone: between 100 micrograms and 300 micrograms;

Cholecalciferol: between 10 micrograms and 50 micrograms;

Calcium: between 2 milligrams and 400 milligrams;

Vitamin E: between 50 milligrams and 270 milligrams;

L-Tryptophan: between 10 milligrams and 500 milligrams; and

Gamma-aminobutyric acid: between 100 milligrams and 750 milligrams.

Menaquinone may be referenced herein as Vitamin K. The Vitamin K may be Vitamin K7, a long-chain menaquinone, or 2-methyl-3-heptaprenyl-1,4-naphthoquinone. Cholecalciferol may be referenced herein as Vitamin D3. Vitamin E may be provided in the form of alpha-tocopherol. Magnesium may be in the form of magnesium glycinate.

In some embodiments, the composition 100 has between 1 milligram and 150 milligrams cannabinol. In some embodiments, the composition 100 has between 15 milligrams and 35 milligrams cannabinol. In some embodiments, the cannabinol content is between 20 milligrams and 30 milligrams. In some embodiments, the cannabinol content is about 25 milligrams.

In some embodiments, the composition 100 has a melatonin content of between 0.1 milligrams and 4 milligrams. In some embodiments, the composition 100 has a melatonin content of between 0.2 milligrams and 2 milligrams. In some embodiments, the composition 100 has a melatonin content of between 0.2 milligrams and 1 milligram. In some embodiments, the composition 100 has a melatonin content of about 0.5 milligrams.

In some embodiments, the composition 100 has a magnesium glycinate content of between 1 milligram and 20 milligrams. In some embodiments, the composition 100 has a magnesium glycinate content of between 1 milligram and 5 milligrams. In some embodiments, the composition 100 has a magnesium glycinate content of between 1 milligram and 3 milligrams. In some embodiments, the composition 100 has a magnesium glycinate content of about 2 milligrams.

In some embodiments, the composition 100 has a menaquinone content of between 150 micrograms and 250 micrograms. In some embodiments, the composition 100 has a menaquinone content of between 175 micrograms and 230 micrograms. In some embodiments, the composition 100 has a menaquinone content of about 200 micrograms. The menaquinone may be in the form of Vitamin K2 MK-7 or long-chain menaquinone, or long chain menaquinone-7.

In some embodiments, the composition 100 has a Vitamin D3 or cholecalciferol content of between 15 micrograms and 35 micrograms. In some embodiments, the composition 100 has a cholecalciferol content of between 20 micrograms and 30 micrograms. In some embodiments, the composition 100 has a cholecalciferol content of about 25 micrograms. In some embodiments, the composition 100 has a cholecalciferol content of about 1000 international units.

In some embodiments, the composition 100 has a calcium content of between 2 milligrams and 200 milligrams. In some embodiments, the composition 100 has a calcium content of between 2 milligrams and 100 milligrams. In some embodiments, the composition 100 has a calcium content of between 2 milligrams and 10 milligrams. In some embodiments, the composition 100 has a calcium content of about 4 milligrams.

In some embodiments, the composition 100 has a Vitamin E content of between 100 milligrams and 150 milligrams. In some embodiments, the composition 100 has a Vitamin E content of between 110 milligrams and 140 milligrams. In some embodiments, the composition 100 has a Vitamin E content of between 120 milligrams and 140 milligrams. In some embodiments, the composition 100 has a Vitamin E content of about 134 milligrams. In some embodiments, the composition 100 has a Vitamin E content of about 200 international units.

In some embodiments, the composition 100 has an L-Tryptophan content of between 10 milligrams and 200 milligrams. In some embodiments, the composition 100 has an L-Tryptophan content of between 10 milligrams and 100 milligrams. In some embodiments, the composition 100 has an L-Tryptophan content of between 30 milligrams and 80 milligrams. In some embodiments, the composition 100 has an L-Tryptophan content of about 50 milligrams.

In some embodiments, the composition 100 has a gamma-aminobutyric acid content of between 100 milligrams and 500 milligrams. In some embodiments, the composition 100 has a gamma-aminobutyric acid content of between 200 milligrams and 400 milligrams. In some embodiments, the composition 100 has a gamma-aminobutyric acid content of between 250 milligrams and 350 milligrams. In some embodiments, the composition 100 has a gamma-aminobutyric acid content of about 300 milligrams.

In some embodiments, the composition 100 is suitable for use as a sleep aid and has the following chemicals or ingredients, in combination: cannabinol, melatonin, magnesium, long-chain menaquinone, cholecalciferol, calcium, Vitamin E, L-Tryptophan, and gamma-aminobutyric acid. Vitamin E may be provided in the form of alpha-tocopherol. Magnesium may be in the form of magnesium glycinate.

As previously described herein, the combination of the compounds or chemicals in combination provides a synergistic effect. The composition 100 has been shown to calm the user's nerves and brain, renormalize synaptic strength, and allow serotonin to move freely. The Applicant has shown that cannabinol or CBN, in combination with the other chemicals in the composition 100, provides dramatic results not seen in other sleep aids. CBN is a non-intoxicating or mildly psychoactive compound created when tetrahydrocannabinol (THC) ages or is exposed to heat or oxygen.

The composition 100 may have the following ingredients in the following ratio from greatest to least, by weight: gamma-aminobutyric acid, Vitamin E, L-Tryptophan, cannabinol, calcium, magnesium glycinate, melatonin, Vitamin K2 MK7, Vitamin D3 or cholecalciferol.

The composition 100 may have the following ingredients in the following ratio from greatest to least, by weight: gamma-aminobutyric acid, Vitamin E, L-Tryptophan, calcium, magnesium glycinate, melatonin, menaquinone in the form of Vitamin K2 MK7, Vitamin D3 or cholecalciferol, cannabinol.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

Moreover, the claims shall be construed such that a claim that recites “at least one of A, B, or C” shall read on a device that requires “A” only. The claim shall also read on a device that requires “B” only. The claim shall also read on a device that requires “C” only.

Similarly, the claim shall also read on a device that requires “A+B”. The claim shall also read on a device that requires “A+B+C”, and so forth.

The claims shall also be construed such that any relational language (e.g. perpendicular, straight, parallel, flat, etc.) is understood to include the recitation “within a reasonable manufacturing tolerance at the time the device is manufactured or at the time of the invention, whichever manufacturing tolerance is greater”.

Those skilled in the art can readily recognize that numerous variations and substitutions may be made in the invention, its use and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

What is claimed is:

1. A stable composition suitable for use as a sleep aid, the composition comprising, in combination:
cannabinol, melatonin, magnesium glycinate, menaquinone, cholecalciferol, calcium, Vitamin E, L-Tryptophan, and gamma-aminobutyric acid.

2. The composition of claim 1, comprising a multiple of the following formula:
Cannabinol: between 10 micrograms and 150 milligrams;
Melatonin: between 0.1 milligrams and 9 milligrams;
Magnesium Glycinate: between 1 milligram and 100 milligrams;
Menaquinone: between 100 micrograms and 300 micrograms;
Cholecalciferol: between 10 micrograms and 50 micrograms;
Calcium: between 2 milligrams and 400 milligrams;
Vitamin E: between 50 milligrams and 270 milligrams;
L-Tryptophan: between 10 milligrams and 500 milligrams; and
Gamma-aminobutyric acid: between 100 milligrams and 750 milligrams.

3. The composition of claim 1, wherein:
the menaquinone is long-chain menaquinone.

4. The composition of claim 3, wherein:
the long-chain menaquinone is 2-methyl-3-heptaprenyl-1,4-naphthoquinone or Vitamin K2 MK-7.

5. The composition of claim 1, wherein:
the Vitamin E is alpha-tocopherol.

6. The composition of claim 2, comprising:
Cannabinol: between 1 milligram and 35 milligrams.

7. The composition of claim 6, comprising:
Cannabinol: between 20 milligrams and 30 milligrams.

8. The composition of claim 2, comprising:
Melatonin: between 0.1 milligrams and 4 milligrams; and
Magnesium Glycinate: between 1 milligram and 20 milligrams.

9. The composition of claim 2, comprising:
Melatonin: between 0.2 milligrams and 2 milligrams; and
Magnesium Glycinate: between 1 milligram and 5 milligrams.

10. The composition of claim 2, comprising:
Menaquinone: between 150 micrograms and 250 micrograms; and
Cholecalciferol: between 15 micrograms and 35 micrograms.

11. The composition of claim 2, comprising:
Menaquinone: between 175 micrograms and 230 micrograms; and
Cholecalciferol: between 20 micrograms and 30 micrograms.

12. The composition of claim 2, comprising:
Calcium: between 2 milligrams and 200 milligrams; and
Vitamin E: between 100 milligrams and 150 milligrams.

13. The composition of claim 2, comprising:
Calcium: between 2 milligrams and 100 milligrams; and
Vitamin E: between 110 milligrams and 140 milligrams.

14. The composition of claim 2, comprising:
L-Tryptophan: between 10 milligrams and 200 milligrams; and
Gamma-aminobutyric acid: between 100 milligrams and 500 milligrams.

15. The composition of claim 2, comprising:
L-Tryptophan: between 10 milligrams and 100 milligrams; and
Gamma-aminobutyric acid: between 200 milligrams and 400 milligrams.

16. The composition of claim 2, comprising:
Cannabinol: between 1 milligrams and 35 milligrams;
Melatonin: between 0.2 milligrams and 2 milligrams;
Magnesium Glycinate: between 1 milligram and 5 milligrams;
Menaquinone: between 175 micrograms and 230 micrograms;
Cholecalciferol: between 20 micrograms and 30 micrograms;
Calcium: between 2 milligrams and 100 milligrams;
Vitamin E: between 110 milligrams and 140 milligrams;
L-Tryptophan: between 10 milligrams and 100 milligrams; and
Gamma-aminobutyric acid: between 200 milligrams and 400 milligrams.

17. The composition of claim 16, comprising:
Cannabinol: between 15 milligrams and 35 milligrams;
Melatonin: between 0.2 milligrams and 1 milligrams;
Magnesium Glycinate: between 1 milligram and 3 milligrams;
Menaquinone: between 175 micrograms and 230 micrograms;
Cholecalciferol: between 20 micrograms and 30 micrograms;
Calcium: between 2 milligrams and 10 milligrams;
Vitamin E: between 120 milligrams and 140 milligrams;
L-Tryptophan: between 30 milligrams and 80 milligrams; and
Gamma-aminobutyric acid: between 250 milligrams and 350 milligrams.

18. The composition of claim 17, comprising:
Cannabinol: (CBN) about 25 milligrams;
Melatonin: about 0.5 milligrams;
Magnesium Glycinate 2 milligrams;
Menaquinone: about 200 micrograms;
Cholecalciferol: about 25 micrograms;
Calcium: about 4 milligrams;
Vitamin E: about 134 micrograms;
L-Tryptophan: about 50 milligrams; and
Gamma-aminobutyric acid: about 300 milligrams.

19. The composition of claim 1, wherein the chemicals are provided in the following ratio from greatest to least, by weight:
gamma-aminobutyric acid, Vitamin E, L-Tryptophan, cannabinol, calcium, magnesium glycinate, melatonin, menaquinone in the form of Vitamin K2 MK7, cholecalciferol.

20. The composition of claim 1, wherein the chemicals are provided in the following ratio from greatest to least, by weight:
gamma-aminobutyric acid, Vitamin E, L-Tryptophan, calcium, magnesium glycinate, melatonin, menaquinone in the form of Vitamin K2 MK7, cholecalciferol, cannabinol.
